(19) United States
(12) Patent Application Publication
(10) Pub. No.: US 2026/0364313 A1
(43) Pub. Date: Nov. 19, 2026

(54) SHEATH INTRODUCER

(71) Applicant: ASAHI INTECC CO., LTD., Seto-shi (JP)

(72) Inventor: Taichi WASHIO, Seto-shi (JP)

(73) Assignee: ASAHI INTECC CO., LTD., Seto-shi (JP)

(21) Appl. No.: 19/660,334

(22) Filed: Apr. 28, 2026

(30) Foreign Application Priority Data
May 13, 2025 (JP) 2025-080396

(51) Int. Cl. A61M 25/06; A61M 25/00; A61M 29/00

(52) U.S. Cl. CPC A61M 25/0662; A61M 25/0045; A61M 25/0053; A61M 25/0054; A61M 29/00; A61M 2205/583

(57) ABSTRACT

A sheath introducer includes a sheath including a lumen extending in an axial direction and a dilator to be inserted into the lumen of the sheath. The sheath includes a first portion positioned on a tip end side and a second portion positioned on a base end side. When the sheath is pressed with a predetermined force in a direction perpendicular to the axial direction, the first portion is more readily deformable than the second portion.

### CROSS-REFERENCE TO RELATED APPLICATIONS

This application claims the benefit of priority to Japanese Application No. 2025-080396, filed May 13, 2025. The disclosure of the prior application is hereby incorporated by reference herein in its entirety.

### TECHNICAL FIELD

The present disclosure relates to a sheath introducer.

### BACKGROUND

Patent Document 1 discloses a sheath introducer including a sheath and a dilator to be inserted into the sheath, the sheath introducer including an interlock that includes a stepped portion of the sheath and a stepped portion of the dilator configured to engage with the stepped portion of the sheath for reducing slipping of the sheath along a surface of the dilator during puncture (so-called tip misalignment).

### PRIOR ART DOCUMENT

### Patent Document

Patent Document 1: JP 2023-513485 T

### SUMMARY

### Technical Problem

A sheath introducer such as the one described in Patent Document 1 may cause problems in that the shape of the interlock interferes with other devices during insertion into a body lumen. When a large force is applied to the sheath introducer as a whole during puncture, the stepped portion of the sheath may ride over the stepped portion of the dilator, causing the interlock to fail to function. Accordingly, conventional sheath introducers leave room for improvement in facilitating integrated manipulation of the sheath and the dilator.

The present disclosure has been made in view of the above circumstances, and provides a sheath introducer that facilitates integrated manipulation of a sheath and a dilator.

### Solution to Problem

The present disclosure provides a sheath introducer including a sheath including a lumen extending in an axial direction of the sheath introducer; and a dilator to be inserted into the lumen of the sheath, in which: the sheath includes a first portion positioned on a tip end side and a second portion positioned on a base end side; and when the sheath is pressed with a predetermined force in a direction perpendicular to the axial direction, the first portion is more readily deformable than the second portion.

### BRIEF DESCRIPTION OF DRAWINGS

FIG. 1 is an overall side view illustrating an example of a sheath introducer according to one embodiment of the present disclosure.

FIG. 2 is a partial side view of the sheath introducer in FIG. 1.

FIG. 3 is a partial cross-sectional view of the sheath introducer in FIG. 1.

FIG. 4 is a partial cross-sectional view of the sheath introducer according to a modification 1.

FIG. 5 is a partial cross-sectional view of the sheath introducer according to a modification 2.

FIG. 6 is a partial cross-sectional view of the sheath introducer according to a modification 3.

### DETAILED DESCRIPTION

Hereinafter, embodiments of the present disclosure are described with reference to the accompanying drawings. The present disclosure is not limited to the embodiments set forth below, and the described embodiments are provided merely for illustrative purposes to explain the technical features of the present disclosure. The shapes and dimensions illustrated in the drawings are provided only to facilitate understanding of the present disclosure and do not accurately reflect actual shapes and dimensions.

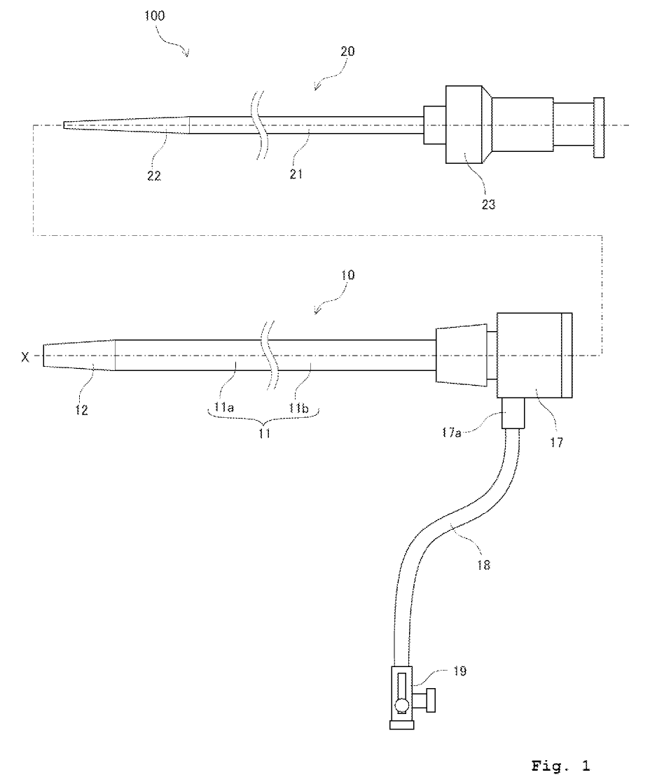

In the present specification, the term “tip end side” refers to a direction along the axial direction of the sheath introducer in which the sheath introducer advances toward a target site. The term “base end side” refers to a direction along the axial direction of the sheath introducer opposite to the tip end side. The term “tip end” refers to an end portion on the tip end side of any given member or part, and the term “base end” refers to an end portion on the base end side of any given member or part. In FIG. 1, the left side of the drawing corresponds to the “tip end side” to be inserted into the body, and the right side of the drawing corresponds to the “base end side” operated by a practitioner.

### Sheath introducer

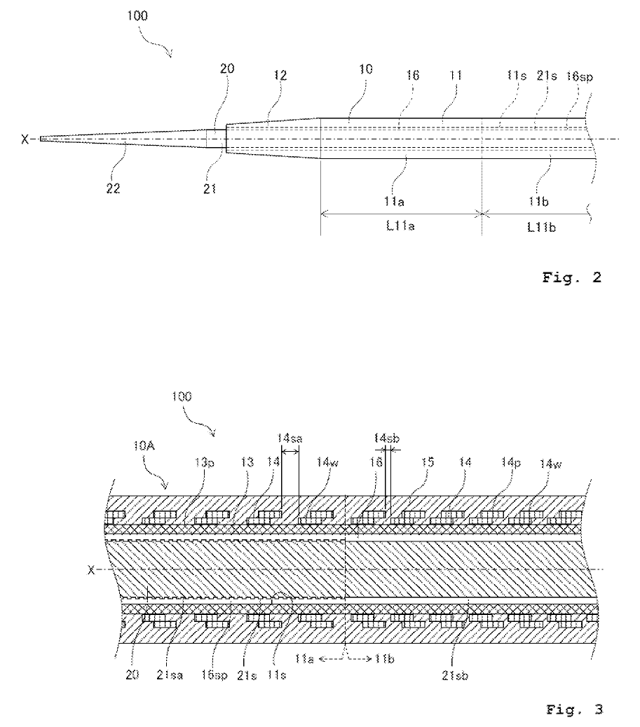

FIG. 1 is an overall side view illustrating an example of a sheath introducer according to one embodiment of the present disclosure. FIG. 2 is a partial side view of a sheath introducer 100 in FIG. 1. The sheath introducer 100 includes a sheath 10 including a lumen 16 extending in an axial direction of the sheath introducer 100, and a dilator 20 to be inserted into the lumen 16 of the sheath 10. FIG. 1 illustrates the sheath 10 and the dilator 20 prior to insertion into the lumen 16 of the sheath 10. FIG. 2 illustrates a state in which the dilator 20 is inserted into the lumen 16 of the sheath 10. In the present embodiment, the axial direction of the sheath introducer 100 refers to a direction along a central axis X of the sheath introducer 100.

As illustrated in FIG. 2, in a state in which the dilator 20 is inserted into the lumen 16 of the sheath 10, a slight gap 16sp is present between an outer peripheral surface 21s of the dilator 20 and an inner peripheral surface 11s of the sheath 10. The gap 16sp has a spacing, for example, in a range of 30 μm to 40 μm.

### Sheath

In the present embodiment, the sheath 10 includes a first portion 11a positioned on the tip end side and a second portion 11b positioned on the base end side. When the sheath 10 is pressed with a predetermined force in a direction perpendicular to the axial direction, the first portion 11a is more readily deformable than the second portion 11b. In the present embodiment, the term “more readily deformable” can be understood as having lower crush resistance or lower pressure resistance. With such a configuration, for example, when a practitioner grips the first portion 11a of the sheath 10, the first portion 11a is slightly deformed in a direction perpendicular to the axial direction and is pressed against the dilator 20. As a result, at the first portion 11a, the outer peripheral surface 21s of the dilator 20 and the inner peripheral surface 11s of the sheath 10 contact each other, thereby making it less likely for the sheath 10 to slide along the outer peripheral surface 21s of the dilator 20. At this time, the lumen 16 of the sheath 10 is not excessively deformed, and a space through which another device can pass is maintained. According to the sheath introducer 100, slipping of the sheath 10 along the surface of the dilator 20 during puncture can be prevented. The sheath introducer 100 according to the present embodiment facilitates integrated manipulation of the sheath 10 and the dilator 20.

In the present embodiment, the sheath 10 includes a main body 11 and a tip end portion 12. The main body 11 is a cylindrical elongated member. The tip end portion 12 is connected to a tip end side of the main body 11. As illustrated in FIGS. 1 and 2, the tip end portion 12 may gradually decrease in diameter from a base end toward a tip end. In the present embodiment, the main body 11 includes the first portion 11a and the second portion 11b. That is, the first portion 11a is a portion positioned between the tip end portion 12 and the second portion 11b.

A length L11a of the first portion 11a in the axial direction can be appropriately set in accordance with the length or the like of the main body 11 of the sheath 10. For example, the length L11a of the first portion 11a may be in a range of 10 mm to 100 mm. With such a configuration, slipping of the sheath 10 along the outer peripheral surface 21s of the dilator 20 during puncture can be more easily prevented.

The length L11a may be in a range of 10 mm to 50 mm, or may be in a range of 10 mm to 30 mm.

A length L11b of the second portion 11b in the axial direction can be appropriately set in accordance with the length or the like of the main body 11 of the sheath 10. For example, the length L11b of the second portion 11b may be in a range of 10 mm to 1500 mm.

FIG. 3 is a partial cross-sectional view of the sheath introducer 100 in FIG. 1. In the example in FIG. 3, the sheath introducer 100 includes a sheath 10A as the sheath 10. The sheath 10A includes an inner layer body 13 and a tubular reinforcing body 14 disposed along an outer periphery 13p of the inner layer body 13. The reinforcing body 14 includes at least one wire 14w. With such a configuration, appropriate rigidity can be imparted to the sheath 10, thereby facilitating advancement of the sheath introducer 100 within a body lumen.

In the sheath 10A illustrated in FIG. 3, a gap 14sa between the adjacent portions of the wire(s) 14w in the first portion 11a is wider than a gap 14sb between the adjacent portions of the wire(s) 14w in the second portion 11b. With such a configuration, a structure can be achieved in which the first portion 11a is more readily deformable than the second portion 11b when the sheath 10 is pressed with a predetermined force in a direction perpendicular to the axial direction. The expression “the gap 14sa between the adjacent portions of the at least one wire 14w in the first portion 11a is wider than the gap 14sb between the adjacent portions of the at least one wire 14w in the second portion 11b” or similar expression can alternatively be expressed as “the density of the reinforcing body 14 in the first portion 11a is lower than the density of the reinforcing body 14 in the second portion 11b”.

The reinforcing body 14 may be a braided body formed by braiding a plurality of wires 14w in a mesh-like form. In the example in FIG. 3, the reinforcing body 14 is a braided body. The reinforcing body 14 may be a multi-strand coil body formed by closely winding a plurality of wires 14w, or a single-strand coil body (single-wire coil) formed by closely winding the single wire 14w. If the reinforcing body 14 is a multi-strand coil body or a single-strand coil body, the gap 14sa between the adjacent wires 14w in the first portion 11a may be wider than the gap 14sb between the adjacent wires 14w in the second portion 11b.

As a material of the wire 14w, for example, stainless steel, tungsten, reinforced plastic, or the like can be used. The cross-sectional shape of the wire 14w may be rectangular or circular. In the example in FIG. 3, the cross-sectional shape of the wire 14w is rectangular.

As illustrated in FIG. 3, the sheath 10 (10A) may include an outer layer body 15 disposed along an outer periphery 14p of the reinforcing body 14. The outer layer body 15 can improve smoothness of the reinforcing body 14.

The outer layer body 15 may be formed of a resin material. The resin material is not particularly limited. Examples of the resin material include, but are not limited to, polyamide, polyamide elastomer, polyester, and polyurethane.

The configuration of the sheath 10 included in the sheath introducer 100 is not limited to the sheath 10A illustrated in FIG. 3. Hereinafter, modifications of the sheath 10 are described.

### Modification 1

FIG. 4 is a partial cross-sectional view of the sheath introducer according to a modification 1. The sheath introducer 100 according to the modification 1 includes a sheath 10B as the sheath 10. In the sheath 10B, the reinforcing body 14 is a multi-strand coil body or a single-strand coil body, and a diameter of the wire 14w present in the first portion 11a is smaller than a diameter of the wire 14w present in the second portion 11b. Except for this difference, the sheath 10B illustrated in FIG. 4 basically has the same configuration as the sheath 10A illustrated in FIG. 3.

With the configuration of the sheath 10B as well, a structure can be achieved in which the first portion 11a is more readily deformable than the second portion 11b when the sheath 10 is pressed with a predetermined force in a direction perpendicular to the axial direction.

In the present embodiment, a diameter of the wire 14w present in the first portion 11a may be represented by an average of the diameter (wire diameter) of all the wires 14w present in the first portion 11a. A diameter of the wire 14w present in the second portion 11b may be represented by an average of the diameter (wire diameter) of all the wires 14w present in the second portion 11b.

### Modification 2

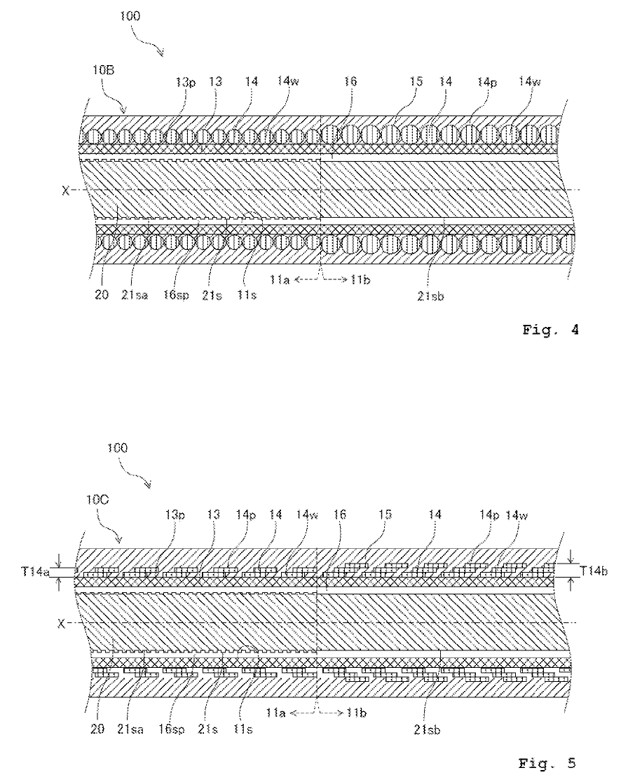

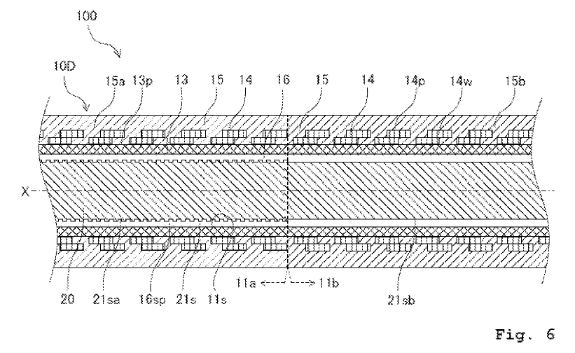

FIG. 5 is a partial cross-sectional view of the sheath introducer according to a modification 2. The sheath introducer 100 according to the modification 2 includes a sheath 10C as the sheath 10. In the sheath 10C, a thickness T14a of the reinforcing body 14 in the first portion 11a is smaller than a thickness T14b of the reinforcing body 14 in the second portion 11b. Except for this difference, the sheath 10C illustrated in FIG. 5 basically has the same configuration as the sheath 10A illustrated in FIG. 3.

With the configuration of the sheath 10C as well, a structure can be achieved in which the first portion 11a is more readily deformable than the second portion 11b when the sheath 10 is pressed with a predetermined force in a direction perpendicular to the axial direction.

As illustrated in FIG. 5, the thickness T14a of the reinforcing body 14 in the first portion 11a refers to a thickness of the reinforcing body 14 in the first portion 11a measured in a cross-section perpendicular to a central axis X and passing through the central axis X. The thickness T14b of the reinforcing body 14 in the second portion 11b refers to a thickness of the reinforcing body 14 in the second portion 11b measured in a cross-section perpendicular to the central axis X and passing through the central axis X.

In the present embodiment, the thickness T14a of the reinforcing body 14 in the first portion 11a may be represented by an average of the thicknesses of the reinforcing bodies 14 at various points in the first portion 11a. The thickness T14b of the reinforcing body 14 in the second portion 11b may be represented by an average of the thicknesses of the reinforcing bodies 14 at various points in the second portion 11b.

### Modification 3

FIG. 6 is a partial cross-sectional view of the sheath introducer according to a modification 3. The sheath introducer 100 according to the modification 3 includes a sheath 10D as the sheath 10. In the sheath 10D, a first material for forming an outer layer body 15a present in the first portion 11a is softer than a second material for forming an outer layer body 15b present in the second portion 11b. Except for this difference, the sheath 10D illustrated in FIG. 6 basically has the same configuration as the sheath 10A illustrated in FIG. 3.

With the configuration of the sheath 10D as well, a structure can be achieved in which the first portion 11a is more readily deformable than the second portion 11b when the sheath 10 is pressed with a predetermined force in a direction perpendicular to the axial direction.

As described above, the outer layer body 15 may be formed of the resin material. Both the first material for forming the outer layer body 15a in the first portion 11a and the second material for forming the outer layer body 15b in the second portion 11b may each be the resin material. With such a configuration, the above-described structure can be more readily achieved.

As illustrated in FIG. 1, the sheath 10 may include a hub 17 on the base end side. A lumen of the hub 17 communicates with the lumen 16 of the main body 11. The hub 17 is provided with a side port 17a communicating with the lumen of the hub 17, and the side port 17a is connected to a three-way stopcock 19 via a tube 18. By means of the three-way stopcock 19, a liquid such as physiological saline can be supplied to the lumen 16 of the main body 11 through the lumen of the hub 17.

### Dilator

In the present embodiment, the dilator 20 includes a main body 21 and a tip end portion 22. The main body 21 is a cylindrical elongated member. The tip end portion 22 is connected to a tip end side of the main body 21. As illustrated in FIGS. 1 and 2, the tip end portion 22 may gradually decrease in diameter from a base end toward a tip end. In the present embodiment, an outer peripheral surface 21s of the dilator 20 corresponds to an outer peripheral surface of the main body 21.

In the present embodiment, a first outer peripheral surface 21sa of the dilator 20 located at a position corresponding to the first portion 11a has a fine uneven structure. With such a configuration, when the first portion 11a is slightly deformed in a direction perpendicular to the axial direction and pressed against the dilator 20, and the outer peripheral surface 21s of the dilator 20 and the inner peripheral surface 11s of the sheath 10 contact each other, adhesion between the outer peripheral surface 21s and the inner peripheral surface 11s can be improved. Accordingly, slipping of the sheath 10 along the outer peripheral surface 21s of the dilator 20 can be further prevented. In the present embodiment, the first outer peripheral surface 21sa of the dilator 20 refers to the outer peripheral surface 21s of the dilator 20 located at a position corresponding to the first portion 11a in the state illustrated in FIG. 2 (i.e., a state in which the dilator 20 is inserted into the lumen 16 of the sheath 10). The same applies to a second outer peripheral surface 21sb of the dilator 20 located at a position corresponding to the second portion 11b.

As illustrated in FIG. 3, the second outer peripheral surface 21sb of the dilator 20 located at the position corresponding to the second portion 11b need not have a fine uneven structure.

A method for providing the fine uneven structure to the first outer peripheral surface 21sa is not particularly limited. For example, one method for providing the fine uneven structure to the first outer peripheral surface 21sa is to roughen a surface of the first outer peripheral surface 21sa by a blasting treatment. Alternatively, for example, the first outer peripheral surface 21sa may be provided with the fine uneven structure by covering the first outer peripheral surface 21sa with a coating material having a fine uneven structure.

An arithmetic average roughness Ra of the first outer peripheral surface 21sa may be greater than an arithmetic average roughness Ra of the second outer peripheral surface 21sb.

A coefficient of static friction μs of the first outer peripheral surface 21sa may be greater than a coefficient of static friction μs of the second outer peripheral surface 21sb.

As illustrated in FIG. 1, the dilator 20 may include a dilator hub 23 on the base end side. The dilator hub 23 is configured to be connectable to the hub 17 of the sheath 10.

In the sheath introducer 100 according to the present embodiment, the first portion 11a and the second portion 11b of the sheath 10 may be configured to be visually distinguishable from each other. With such a configuration, a practitioner can easily recognize the first portion 11a. Accordingly, by gripping the first portion 11a of the sheath 10, the practitioner can prevent slipping of the sheath 10 along the outer peripheral surface 21s of the dilator 20.

The method for configuring the first portion 11a and the second portion 11b to be visually distinguishable is not particularly limited. For example, the first portion 11a and the second portion 11b may be configured to be visually distinguishable by using different colorants added to the first material for forming the outer layer body 15a in the first portion 11a and to the second material for forming the outer layer body 15b in the second portion 11b. Alternatively, for example, the first portion 11a and the second portion 11b may be configured to be visually distinguishable by providing different markings on the outer layer body 15a in the first portion 11a and on the outer layer body 15b in the second portion 11b.

### Method for producing sheath introducer

The sheath introducer 100 according to the present embodiment can be produced, for example, by the following method. In the following, a method for producing the sheath introducer 100 illustrated in FIG. 3 is described as an example.

First, a resin material A is applied to an outer periphery of a core wire having a predetermined outer diameter to form the inner layer body 13. The resin material A is, for example, polytetrafluoroethylene (PTFE). Next, the tip end portion 12 is formed on a tip end side of the inner layer body 13 using a resin material B. The resin material B is, for example, polyurethane. Next, the reinforcing body 14 is wound along the outer periphery 13p of the inner layer body 13. In this process, the reinforcing bodies 14 are arranged such that the gap 14sa between the adjacent wires 14w in the first portion 11a on the tip end side is wider than the gap 14sb between the adjacent wires 14w in the second portion 11b on the base end side. Next, the outer layer body 15 is formed along the outer periphery 14p of each reinforcing body 14. Heat treatment is then performed in this state. After cooling, the core wire is pulled out. The sheath 10 is obtained by connecting the hub 17 to the base end side. Finally, the sheath introducer 100 is obtained by inserting the dilator 20 into the sheath 10. In the dilator 20, the first outer peripheral surface 21sa corresponding to the first portion 11a of the sheath 10 has a fine uneven structure.

The present disclosure provides the following.

### Disclosure 1

A sheath introducer including:
a sheath including a lumen extending in an axial direction of the sheath introducer; and
a dilator to be inserted into the lumen of the sheath, in which:
   the sheath includes a first portion positioned on a tip end side and a second portion positioned on a base end side, and;
   when the sheath is pressed with a predetermined force in a direction perpendicular to the axial direction, the first portion is more readily deformable than the second portion.

According to the above (Disclosure 1), integrated manipulation of the sheath and the dilator can be facilitated.

### Disclosure 2

The sheath introducer according to the above (Disclosure 1), in which the sheath includes an inner layer body and a tubular reinforcing body disposed along an outer periphery of the inner layer body, the reinforcing body including a wire.

According to above (Disclosure 2), appropriate rigidity can be imparted to the sheath, thereby facilitating advancement of the sheath introducer within a body lumen.

### Disclosure 3

The sheath introducer according to the above (Disclosure 1 or 2), in which a gap between the adjacent wires in the first portion is wider than a gap between the adjacent wires in the second portion.

According to the above (Disclosure 3), a structure can be achieved in which the first portion is more readily deformable than the second portion when the sheath is pressed with a predetermined force in a direction perpendicular to the axial direction.

### Disclosure 4

The sheath introducer according to any of the above (Disclosures 1 to 3), in which a diameter of the wire in the first portion is smaller than a diameter of the wire in the second portion.

According to the above (Disclosure 4), a structure can be achieved in which the first portion is more readily deformable than the second portion when the sheath is pressed with a predetermined force in a direction perpendicular to the axial direction.

### Disclosure 5

The sheath introducer according to any of the above (Disclosures 1 to 4), in which a thickness of the reinforcing body in the first portion is smaller than a thickness of the reinforcing body in the second portion.

According to the above (Disclosure 5), a structure can be achieved in which the first portion is more readily deformable than the second portion when the sheath is pressed with a predetermined force in a direction perpendicular to the axial direction.

### Disclosure 6

The sheath introducer according to any of the above (Disclosures 1 to 5), in which:
the sheath includes an outer layer body disposed along an outer periphery of the reinforcing body; and
a first material for forming the outer layer body in the first portion is softer than a second material for forming the outer layer body in the second portion.

According to the above (Disclosure 6), a structure can be achieved in which the first portion is more readily deformable than the second portion when the sheath is pressed with a predetermined force in a direction perpendicular to the axial direction.

### Disclosure 7

The sheath introducer according to any of the above (Disclosures 1 to 6), in which, in a state in which the dilator is inserted into the lumen of the sheath, an outer peripheral surface of the dilator located at a position corresponding to the first portion has a fine uneven structure.

According to the above (Disclosure 7), when the outer peripheral surface of the dilator and the inner peripheral surface of the sheath contact each other, adhesion between the outer peripheral surface of the dilator and the inner peripheral surface of the sheath can be improved.

### Disclosure 8

The sheath introducer according to any of the above (Disclosures 1 to 7), in which a length of the first portion in the axial direction is in a range of 10 mm to 100 mm.

According to the above (Disclosure 8), slipping of the sheath along the outer peripheral surface of the dilator during puncture can be more easily prevented.

### Disclosure 9

The sheath introducer according to any of the above (Disclosures 1 to 8), in which the first portion and the second portion are visually distinguishable from each other.

According to the above (Disclosure 9), a practitioner can easily recognize the first portion.

As described above, the sheath introducer according to the present disclosure has been explained with reference to the drawings. The present disclosure is not limited to the above-described embodiments, and various modifications can be made. Each of the constituent elements of the above-described embodiments and modifications 1 to 3 may be combined with one another insofar as they are not technically inconsistent.

1. A sheath introducer, comprising:
a sheath including a lumen extending in an axial direction of the sheath introducer; and
a dilator configured to be inserted into the lumen of the sheath, wherein:
the sheath includes a first portion positioned on a tip end side and a second portion positioned on a base end side; and
when the sheath is pressed with a predetermined force in a direction perpendicular to the axial direction, the first portion is more readily deformable than the second portion.

2. The sheath introducer according to claim 1, wherein the sheath includes an inner layer body and a tubular reinforcing body disposed along an outer periphery of the inner layer body, the reinforcing body including at least one wire.

3. The sheath introducer according to claim 2, wherein a gap between adjacent portions of the at least one wire in the first portion is wider than a gap between adjacent portions of the at least one wire in the second portion.

4. The sheath introducer according to claim 2, wherein a diameter of the at least one wire in the first portion is smaller than a diameter of the at least one wire in the second portion.

5. The sheath introducer according to claim 2, wherein a thickness of the reinforcing body in the first portion is smaller than a thickness of the reinforcing body in the second portion.

6. The sheath introducer according to claim 2, wherein
the sheath includes an outer layer body disposed along an outer periphery of the reinforcing body; and
a first material for forming the outer layer body in the first portion is softer than a second material for forming the outer layer body in the second portion.

7. The sheath introducer according to claim 1, wherein, in a state in which the dilator is inserted into the lumen of the sheath, an outer peripheral surface of the dilator located at a position corresponding to the first portion has a fine uneven structure.

8. The sheath introducer according to claim 1, wherein a length of the first portion in the axial direction is in a range of 10 mm to 100 mm.

9. The sheath introducer according to claim 1, wherein the first portion and the second portion are visually distinguishable from each other.
